(19) United States
(12) Patent Application Publication
(10) Pub. No.: US 2006/0134216 A1
(43) Pub. Date: Jun. 22, 2006

(54) Enteric film coating composition containing enteric polymer micronized with detackifier

(76) Inventors: Thomas P. Farrell, Warrington, PA (US); Kurt Fegely, Gilbertsville, PA (US); Ali Rajabi-Siahboomi, Lansdale, PA (US); George Reyes, Perkiomenville, PA (US); Budhi Simon, Harleysville, PA (US); Cara Young, Blue Bell, PA (US)

Correspondence Address: LUCAS & MERCANTI, LLP, 475 PARK AVENUE SOUTH, 15TH FLOOR, NEW YORK, NY, 10016, US

(21) Appl. No.: 11/305,900

(22) Filed: Dec. 16, 2005

Related U.S. Application Data
(60) Provisional application No. 60/637,083, filed on Dec. 17, 2004.

(51) Int. Cl. A61K 9/24

(52) U.S. Cl. 424/472

(57) ABSTRACT

Dry, enteric, film-coating compositions and aqueous dispersions containing the same are disclosed. When applied to orally-ingestible substrates such as oral solid dosage forms, the film coatings are capable of preventing the substrates from disintegrating in media with pH values from about 1 to about 4.5 or higher values. One preferred film-coating composition contains a micronized intermediate comprised of an acrylic resin and talc. Advantageously and surprisingly, the preferred film-coating composition does not contain an alkalizing agent.

### CROSS-REFERENCE TO RELATED APPLICATIONS

This application claims the benefit of priority from U.S. Provisional Application Ser. No. 60/637,083, filed Dec. 17, 2004, the contents of which are incorporated herein by reference.

### BRIEF DESCRIPTION OF THE INVENTION

This invention is directed to a dry, fully-formulated, enteric, film-coating composition, which when applied in an aqueous dispersion to coat orally-ingestible substrates, is capable of preserving said orally-ingestible substrates from disintegration in media with pH values from about 1 to about 4.5 or higher values. One preferred film-coating composition contains a micronized intermediate comprised of an acrylic resin and talc. Advantageously and surprisingly, the preferred film-coating composition does not contain an alkalizing agent. Methods are disclosed for the production of: 1) the micronized intermediate; 2) dry, fully-formulated film-coating compositions comprising the intermediate; 3) aqueous dispersions containing the film-coating compositions; and 4) orally-ingestible substrates coated with the inventive aqueous dispersions.

### BACKGROUND OF THE INVENTION

It is well-known that the pH of the stomach may vary between about 1 and about 4.5 based upon a number of factors. For example, the pH of the stomach may be raised from about pH 1 in the fasted state to about pH 4.5 or higher in the fed state. Also, certain drugs are capable of raising the pH of the stomach, again from about pH 1 to about pH 4.5 or higher based on the pharmacological action of the drug. Among the drugs capable of raising the pH of the stomach is a class of drugs known as proton pump inhibitors (PPIs) or 2-[[(2-pyridinyl)methyl]-sulfinyl]benzimidazoles, which are known to have anti-ulcer activity. Examples of drugs in this class are omeprazole, lansoprazole, pantoprazole, rabeprazole and esomeprazole. While these drugs have well-established therapeutic effects, they are also known to be prone to rapid degradation in acidic media. For example, omeprazole has a half-life of less than ten minutes in aqueous solution at pH values under 4.0 (U.S. Pat. No. 6,623,759).

It is often desirable to design an orally-ingestible dosage form such that it will not disintegrate or dissolve substantially in the stomach but will, subsequently, quickly dissolve upon entering the small intestines. This is particularly true in the case of PPIs, since they are known to degrade substantially in the stomach, even at the higher end of the pH range typically encountered therein (i.e. about 4.5 or greater). Therefore, it is essential that the PPI dosage forms are preserved as they pass through the stomach but dissolve rapidly in the small intestines to achieve maximum bioavailability. PPI products have been formulated with this principle in mind (U.S. Pat. No. 6,207,198; U.S. Pat. No. 6,569,457; and U.S. Pat. No. 6,623,759); however, the coatings used in dosage form development are often laboriously formulated in stepwise processes.

U.S. Pat. No. 6,420,473 describes a non-toxic, edible, enteric film coating, dry powder composition comprised of an acrylic resin, an alkalizing agent and a detackifier. This fully-formulated system, marketed under the trade name Acryl-EZE®, simplifies the coating process, since the preparation of a coating dispersion requires only the addition of the fully-formulated system to water in one-step versus the time-consuming, multi-step processes previously known in the field. The alkalizing agent is an essential component in the '473 formulations, because it partially neutralizes the acrylic resin thereby allowing the formation of a homogeneous aqueous dispersion, without the formation of coagulum, when the dry powders are added to water.

### SUMMARY OF THE INVENTION

According to one aspect of the invention, there is provided a dry, enteric, film-coating composition, which, in most cases does not include an alkalizing agent, but still can be homogeneously dispersed in water, substantially without the formation of coagulum. Consequently, the inventive film-coating composition is also capable of being film-coated onto orally-ingestible substrates and substantially preserving them from disintegration in media with pH values from about 1 to about 4.5 or higher. The inventive dry, enteric, film-coating composition includes a micronized blend of an enteric polymer and a detackifier, wherein the enteric polymer is micronized in the presence of a portion of the detackifier.

Other aspects of the invention include methods of preparing and using the film-coating compositions as well as aqueous dispersions containing the same. Still further aspects include pharmaceutical substrates coated therewith.

### DESCRIPTION OF THE INVENTION

In one aspect of the invention, the inventive, dry composition is comprised of an enteric polymer, a detackifier and, optionally a plasticizer. The enteric polymer may be any polymer capable of forming a coating on orally-ingestible substrates, which will not dissolve in low pH environments, for example from about pH 1 to about pH 4.5 or higher. Suitable enteric polymers include, for example, acrylic resins, polyvinylacetate phthalate, cellulose acetate phthalate, hydroxypropylmethylcellulose phthalate and any other enteric polymers useful for coating orally-ingestible substrates. See also commonly-assigned U.S. Pat. No. 5,733,575, the disclosure of which is incorporated herein by reference which discloses enteric formulations based on micronized PVAP. Acrylic resins, however, are preferred enteric polymers. The acrylic resin comprises: 1) from 20 to 85 percent by weight of at least one alkyl acrylate or alkyl methacrylate moiety; 2) from 80 to 15 percent by weight of at least one vinyl or vinylidene moiety having a carboxylic acid group; and 3) from 0 to 30 percent by weight of at least one other vinyl or vinylidene moiety copolymerizable with (1) and (2). A non-limiting list of suitable acrylic resins includes, for example, Eudragit® L100, Eudragit L100-55 and Eudragit S100. Combinations/mixtures of acrylic resins are also contemplated. Preferred acrylic resins are copolymers of methacrylic acid and methyl methacrylate; and methacrylic acid and ethyl acrylate. The most preferred acrylic resin is a copolymer of ethyl acrylate and methacrylic acid. One example of the most preferred acrylic resin is Eudragit L100-55. Preferably, the enteric polymer comprises from about 40 to about 70% of the dry film coating composition. More preferably, the enteric polymer comprises from about 45 to about 65% of the dry film coating composition.

In most aspects of the invention, the detackifier has two primary functions. First, a portion or all of the detackifier is blended with the acrylic resin and then micronized to obtain an intimate mixture of the two components. As will be disclosed in more detail later, the micronization of this preblend allows the artisan to obtain a film coating dispersion with a minimum amount of coagulum. Without wishing to be bound by theory, it is postulated that, in this capacity, the detackifier physically restricts intermolecular and intramolecular association of the acrylic resin thereby reducing its ability to agglomerate. The second primary function of the detackifier is to reduce the incidence of substrate-to-substrate sticking during the film coating process.

The detackifier may be any inorganic or organic species capable of physically restricting the intermolecular or intramolecular association of the enteric polymer in the dry or aqueously-dispersed state. The detackifier may be talc, silicon dioxide, silca gel, fumed silica, kaolin, glyceryl monostearate or mixtures thereof. Talc is the preferred detackifier. Preferably, the detackifier comprises about 1-33% of the micronized acrylic resin/talc preblend and about 0.1 to about 35% of the final dry film-coating composition.

A first portion of the detackifier may be incorporated in the micronized preblend and a second portion in the final film-coating formulation after the micronization step. As will be appreciated by those of ordinary skill, the detackifier included in the micronized preblend can be the same as or different from the remainder of the detackifier used in the compositions of the present invention. For purposes of describing the process for making the inventive compositions, reference is made to a “first” detackifier used for preparing the pre-blend and a “second” detackifier added thereafter usually in combination with other film coating ingredients. The preferred ratio of enteric polymer to detackifier in the micronized preblend is from 2:1 to 99:1. The most preferred ratio of enteric polymer to detackifier in the micronized preblend is from 3:1 to 20:1.

Micronization of the enteric polymer alone does not yield a product that is suitable for the purposes of this invention. Instead, it has been surprising found that when the preferred enteric polymers are micronized with a sufficient amount of a detackifier, the advantageous properties are realized as compared to that obtained when standard mixing techniques are employed. While Applicants are not bound by theory, it is believed that the combination of forces which act upon the enteric polymer and detackifier causing a reduction of particle size during micronization also cause a somewhat unique combining of the ingredients. The micronization process thus advantageously transforms the separate ingredients into a mixture which has properties that are different from those observed when the combination of ingredients are not micronized. If desired, when the second detackifier added to the final film-coating formulation after the micronization step, it is preferably present in amount of from 0 to about 15% of the overall weight of the final film-coating formulation. Regardless of whether the detackifier is added completely as part of the micronized pre-blend or divided into micronized and non-micronized portions, the most preferred overall amount of detackifier in the final film-coating formulation is about 15-30%.

The compositions of the present invention will also preferably include a plasticizer. The plasticizer may be any of those which have been used successfully with acrylic resins. Preferred plasticizers are triethylcitrate, triacetin, polyethylene glycol (PEG) of varying molecular weights, propylene glycol, glyceryl triacetate, acetyltriethylcitrate, dibutyl sebacate, diethylphthalate, dibutylphthalate, glycerin, castor oil, copolymers of propylene oxide and ethylene oxide or mixtures thereof. Of these plasticizers, solid plasticizers are most preferred since they have a lesser tendency to promote agglomeration than liquid plasticizers. Combinations of liquid and solid plasticizers may be used. PEG 3350 and PEG 8000 are particularly preferred plasticizers. The preferred amount of plasticizer in the film coating formulation is from about 5 to about 25%. In some aspects of the invention, the plasticizer may be added, all or in part, to the dry film-coating composition. In alternative and some preferred aspects of the invention, the plasticizer is added separately, all or in part, to the film coating dispersion resulting from the addition of the dry powder composition containing the micronized enteric polymer and detackifier to water.

Optional components of the film-coating composition include flow aids, surfactants, anti-agglomerating agents, secondary film-formers and pigments. The flow aid allows the fully-formulated powder to readily flow during blending, packaging, dispersion preparation and other manipulations. Advantageously, the flow aid also can absorb liquid plasticizers, which reduces the tendency of the film-coating compositions to agglomerate. The preferred flow aids are fumed or fine particle grades of silica such as Cab-O-Sil® supplied by Cabot, Inc. and Syloid® supplied by W.R. Grace. The preferred amount of flow aid is from 0 to about 10%. The most preferred amount of flow aid is from 1 to about 7%. The surfactant may be an ionic or non-ionic surfactant. Preferred surfactants are polysorbates such as Polysorbate 80, sodium lauryl sulfate, dioctylsodium sulfosuccinate and mixtures thereof. The preferred level of surfactant is from 0 to about 3%. The anti-agglomerating agent may be any substance capable of preventing agglomeration of the inventive film-coating composition in the dry state. The preferred anti-agglomerating agent is kaolin. The preferred level of the anti-agglomerating agent is from 0 to about 40%.

The secondary film former may be any polymer capable of raising the viscosity of the inventive aqueous dispersions or increasing the film strength of the inventive film coatings. Preferred secondary film-formers are xanthan gum, sodium alginate, propylene glycol alginate, hydroxypropylmethyl cellulose (HPMC), hydroxyethyl cellulose (HEC), sodium carboxymethylcelullose (NaCMC), polyvinylpyrrolidone (PVP), Konjac flour, carrageenan or mixtures thereof. The preferred level of the secondary film-former is 0 to about 20%.

The pigment may be an FD&C or a D&C lake, titanium dioxide, iron oxides, riboflavin, circumin, carmine 40, annatto, insoluble or soluble dyes, pearlescent pigments based on mica and/or titanium dioxide, magnesium carbonate, talc, pyrogenic silica, iron oxides, channel black, riboflavin, or mixtures thereof. The preferred amount of pigment is from 0 to about 20%. The plasticizer and optional components may be added, all or in part, to the dry film-coating composition; and, all or in part, to the film coating dispersion resulting from the addition of the dry powder composition to water.

Micronization of enteric polymer/detackifier preblends can be achieved by using standard processing equipment known to reduce the particle sizes of powders. The micronized preblend is obtained by first mixing the polymer and detackifier using standard powder mixing equipment to obtain a homogeneous mixture, which does not exhibit a significant reduction in particle size, and then micronizing the mixture in a separate operation. Optionally, mixing and micronization of the enteric polymer and detackifier may occur in operation in suitable micronization equipment. Examples of suitable mixing equipment which are useful to achieve a homogeneous mixture are Paterson-Kelly “V-blenders” as well as blenders manufactured by Readco and Ruberg. For small-scale mixing, a food processor may be utilized. Suitable micronization equipment includes mechanical and pneumatic milling systems. The average particle size of the preblend should be in the range of 0.1 to 50 microns (a micron is equivalent to a micrometer). Preferably, the particle size of the preblend should be in the range of 1 to 30 microns. Most preferably, the average particle size of the preblend should be in the range of 5 to 15 microns.

The micronized preblends are then formulated into complete film-coating systems by adding a plasticizer, and optionally one or more of a second detackifier, a flow aid, an anti-agglomerating agent, a secondary film-former, a pigment or other ingredients known to those of ordinary skill in the art. Again, any blender capable of producing a homogeneous mixture may be utilized. Examples of suitable mixing equipment that are useful to achieve a homogeneous mixture are Paterson-Kelly “V-blenders” as well as blenders manufactured by Readco and Ruberg. For small-scale mixing, a food processor may be utilized.

In another aspect of the invention, there is provided aqueous dispersions suitable for film coating oral solid dosage forms and the like. The dispersions are prepared by adding the complete film-coating system into water with agitation. Alternatively, if desired, the optional plasticizer, flow aid and/or pigment may be added separately to the aqueous dispersion after the micronized preblend has been dispersed. Typically, the concentration of the film-coating system in water is from about 10 to about 20% (w/w). Most preferably, the concentration of the film-coating system in water is from about 15 to about 20%. Care should be exercised to add the complete film-coating system or optional additives to water at a rate slow enough to avoid clumping of the product. Once the complete film-coating system is added to water and a homogeneous dispersion is obtained, the dispersion is passed through a 60-mesh screen to remove any residual agglomerates or coagulum (typically less than about 3%, and preferably less than about 1% dry weight that may have been formed upon dispersion.

The aqueous dispersions may be coated on orally-ingestible dosage forms using any of the standard film coating equipment that are known in the field. In most aspects of the invention, the coating is applied until weight gains of from about 5 to about 30 % are achieved. A non-limiting list of suitable equipment includes film coating pans manufactured by O'Hara and Thomas and fluid bed coaters manufactured by Glatt and Niro.

Subcoats may be coated onto orally-ingestible tablets prior to the application of the inventive film-coating composition in order to improve the mechanical strength of the substrates or otherwise impart some beneficial property, using techniques and amounts well known to those of ordinary skill. The weight of the subcoats applied may be from about 0.1 to about 20% of the starting weight (i.e. 0.1 to 20% weight gain) of the orally-ingestible substrates. Topcoats may also be coated onto orally-ingestible substrates already coated with the inventive film-coating system in order to further enhance the aesthetic appearance or impart some additional property such as flavor. The weight of the topcoats may be from about 0.1 to about 20% of the starting weight (i.e. 0.1 to 20% weight gain) of the orally-ingestible substrates coated with the inventive film-coating compositions and optional subcoats. The orally-ingestible substrates may be any solid substance capable of being ingested orally and imparting a therapeutic effect or health benefit. Examples of orally-ingestible substrates include tablets, caplets, beads, granules and capsules containing one or more active ingredients. In some preferred embodiments, the active ingredients included in the substrates are selected from among proton pump inhibitors (PPIs) or 2-[[(2-pyridinyl)methyl]-sulfinyl]benzimidazoles, such as omeprazole, lansoprazole, pantoprazole, 5 rabeprazole and esomeprazole. It will be understood by those of ordinary skill, however, that the invention is not limited to specific pharmaceutically active ingredients and that it is contemplated that a myriad of pharmaceutically active ingredients can be incorporated into dosage forms containing the inventive coatings described herein.

For purposes of illustration and not limitation, a few of the presently preferred, fully formulated dry enteric film-coating compositions are described below:

Micronized Preblend

| | Preferred Range | Most Preferred Range |
|---|---|---|
| Ingredient | (wt %) | (wt %) |
| Enteric polymer | 67-99 | 75-95 |
| Detackifier | 1-33 | 5-25 |
| Enteric polymer:Detackifier | 2:1-99:1 | 3:1-20:1 |
| Ratio | | |

Dry, Enteric Film-Coating Composition

| Ingredient | Preferred Range (wt %) |
|---|---|
| Enteric polymer* | 40-70 |
| Detackifier** | 0.1-35 |
| Plasticizer | 5-2530 |
| Flow aid | 0-10 |
| Surfactant | 0-3 |
| Anti-agglomerating agent | 0-40 |
| Secondary film-former | 0-20 |
| Pigment | 0-20 |

*Previously micronized with at least a portion of the detackifier.
**A portion of which was previously micronized with the enteric polymer.

### EXAMPLES

### Example 1

To a food processor were added a micronized preblend of Eudragit L100-55 and talc in a 4:1 ratio (75 parts; mean particle size=8 microns), PEG 3350 (18 parts), Syloid 244FP silica (2 parts) and incremental talc (5 parts). The resulting mixture was blended for five minutes. An aqueous dispersion was subsequently prepared by adding 15 parts of the blended composition to 85 parts of deionized water (15% solids suspension) with stirring. The resulting aqueous dispersion was then passed through a 60 mesh screen, and only a very small amount of retained particles (<2% wet weight with respect to the film coating composition) was observed. The screened aqueous dispersion was subsequently coated onto a mixed charge of placebos and aspirin, which had been previously subcoated with Opadry YS-1-7027 to a 4% theoretical weight gain, using an O'Hara Labcoat I film coating pan with a 12″ insert. During the coating run, the bed temperature was maintained at 30 to 33.5° C. Samples were removed periodically from the coating pan at estimated theoretical weight gains of 10, 12 and 14%. Aspirin and placebo tablets coated to 10, 12 and 14% weight gain were separately placed in a disintegration bath containing sodium acetate buffer at pH 4.5. None of the tablets disintegrated during the two hour exposure period. Acid uptake values of the coated aspirin (% increase in tablet weight after immersion in the disintegration bath) were 4.2, 4.4 and 4.3% at 10, 12 and 14% weight gain, respectively. Acid uptake values of coated placebos were 6.2, 5.6 and 5.1% at 10, 12 and 14% weight gain, respectively. For the coated placebos, the acid uptake values decreased with increasing weight gain.

### Example 2 Comparative

Eudragit L100-55 (60 parts) and talc (15 parts), both used as received from the respective suppliers, were premixed in a food processor for five minutes. To this mixture were added PEG 3350 (18 parts), Syloid 244FP silica (2 parts) and incremental talc (5 parts). The mixture was stirred for an additional five minutes. Fifteen parts of the resulting mixture was then added to 85 parts of deionized water with stirring. After stirring for forty minutes, a large amount of coagulum was observed and ultimately retained on a 60 mesh screen. The dispersion was deemed to be uncoatable. Conclusion: A film-coating system based on an Eudragit L100-55/talc preblend, prepared by conventional blending (i.e. without particle size reduction), can not be adequately dispersed in water nor coated.

### Example 3 Comparative

To a food processor were added micronized Eudragit L100-55 (60 parts, mean particle size=8 microns), talc (20 parts) used as received from the supplier, PEG 3350 (18 parts) used as received from the supplier, and Syloid 244FP silica (2 parts) used as received from the supplier. The mixture was blended for five minutes. An aqueous dispersion was subsequently prepared by adding 15 parts of the blended compositions to 85 parts of deionized water (15% solids suspension) with stirring. After stirring for forty minutes, the resulting aqueous dispersion was then passed through a 60 mesh screen. Only a very small amount of particles (<0.5% dry weight with respect to the dry film coating composition) were retained on the screen. The screened aqueous dispersion was then coated onto placebo tablets, which had been previously sub-coated with Opadry YS-1-7027 to a 4% theoretical weight gain, using an O'Hara Labcoat I coater with a 10″ pan insert. The coating run was stopped after a few minutes due to gelling of the dispersion in the line that causing complete line blockage. Conclusion: A fully formulated dry film-coating system based on micronized Eudragit L100-55 and conventional talc (i.e. talc used as received from the supplier) with PEG 3350 as the only plasticizer can form a good aqueous dispersion. However, this dispersion cannot be applied due to tendency of gelling in the tubing during coating run.

### Examples 4-7

In Examples 4-7, a micronized Eudragit L100-55/talc pre-blend was again utilized; however, the plasticizers were added separately to the aqueous dispersions rather than in the formulations containing the micronized pre-blend. The ratio of components used in these examples is provided in the following table:

| | Exam- | Exam- | Exam- | Exam- |
|---|---|---|---|---|
| | ple 4 | ple 5 | ple 6 | ple 7 |
| Pre-mixed Components | | | | |
| Micronized Eudragit L100- | 10.71 | 10.71 | 11.25 | 11.25 |
| 55/Talc (80/20; w/w) | | | | |
| Talc (used as is from supplier) | — | — | 1.05 | 1.95 |
| Separately-added Plasticizers | | | | |
| Propylene glycol | 4.29 | — | — | — |
| Triacetin | — | 4.29 | — | — |
| Triethyle citrate | — | — | 2.7 | — |
| Polyethylene glycol 8000 | — | — | — | 1.8 |

### Examples 4 and 5

10.71 parts of the micronized preblend of Eudragit L100-55 and talc in a 4:1 ratio (mean particle size=8 microns) were added to 85 parts water and stirred for 2 minutes. To this dispersion 4.29 parts of either propylene glycol (Example 4) or triacetin (Example 5) were added as a plasticizing agent and stirred for 30 minutes. The resulting aqueous dispersion was then passed through a 60 mesh screen, and a very small amount of retained particles was observed on the screen.

The screened aqueous dispersion was subsequently coated onto placebo cores which had previously been subcoated with Opadry YS-1-7027 to a 4% theoretical weight gain using an O'Hara Labcoat I film coating pan with a 19″ insert. During the coating run, the bed temperature was maintained at 30-35° C. Samples were removed periodically from the coating pan at estimated theoretical weight gains of 10, 12, and 14%. Samples were separately placed for 2 hours in a disintegration bath containing sodium acetate at pH 4.5. None of the tablets exhibited signs of bloating, cracks, or fissures.

### Example 6

To a food processor were added the micronized preblend of Eudragit L100-55 and talc in a 4:1 ratio (11.25 parts; mean particle size=8 microns), and 1.05 parts incremental talc. The resulting mixture was blended for 5 minutes. An aqueous dispersion was subsequently prepared by adding this preblended composition to 85 parts deionized water with stirring. To this dispersion, 2.7 parts triethyl citrate was added as a plasticizing agent and stirred for 30 minutes. The resulting aqueous dispersion was then passed through a 60 mesh screen, and a very small amount of retained particles was observed on the screen. The screened aqueous dispersion was subsequently coated onto placebo cores which had previously been subcoated with Opadry YS-1-7027 to a 4% theoretical weight gain using an O'Hara Labcoat I film coating pan with a 19″ insert. During the coating run, the bed temperature was maintained at 30-35° C. Samples were removed periodically from the coating pan at estimated theoretical weight gains of 10, 12, and 14%. Samples were separately placed for 2 hours in a disintegration bath containing sodium acetate at pH 4.5. None of the tablets exhibited signs of bloating, cracks, or fissures.

### Example 7

To a food processor were added the micronized preblend of Eudragit L100-55 and talc in a 4:1 ratio (11.25 parts; mean particle size=8 microns), and 1.95 parts incremental talc. The resulting mixture was blended for 5 minutes. An aqueous dispersion was subsequently prepared by adding the preblended composition to 85 parts deionized water with stirring. To this dispersion, 1.8 parts polyethylene glycol 8000 was added as a plasticizing agent and stirred for 30 minutes. The resulting aqueous dispersion was then passed through a 60 mesh screen, and a very small amount of retained particles was observed on the screen. The screened aqueous dispersion was subsequently coated onto placebo cores which had previously been subcoated with Opadry YS-1-7027 to a 4% theoretical weight gain using an O'Hara Labcoat I film coating pan with a 19″ insert. During the coating run, the bed temperature was maintained at 30-35° C. Samples were removed periodically from the coating pan at estimated theoretical weight gains of 10, 12, and 14%. Samples were separately placed for 2 hours in a disintegration bath containing sodium acetate at pH 4.5. None of the tablets exhibited signs of bloating, cracks, or fissures.

### Example 8

In this example one plasticizer (PEG 8000) was included as part of the dry formulation with the micronized Eudragit L100-55/talc preblend and a second plasticizer (triacetin) was added separately to the aqueous dispersion.

To a food processor were added the micronized preblend of Eudragit L100-55 and talc in a 4:1 ratio (82.4 parts; mean particle size=8 microns), PEG 8000 (9.9 parts), and 7.7 parts incremental talc. The resulting mixture was blended for 5 minutes. An aqueous dispersion was subsequently prepared by adding 13.65 parts of the blended composition to 85 parts deionized water with stirring. To this dispersion, 1.35 parts triacetin was added as an additional plasticizing agent and stirred for 30 minutes. The resulting aqueous dispersion was then passed through a 60 mesh screen, and a very small amount of retained particles was observed on the screen. The screened aqueous dispersion was subsequently coated onto placebo cores which had previously been subcoated with Opadry YS-1-7027 to a 4% theoretical weight gain using an O'Hara Labcoat I film coating pan with a 19″ insert. During the coating run, the bed temperature was maintained at 30-35° C. Samples were removed from the coating pan at an estimated theoretical weight gain of 12%. Samples were placed for 2 hours in a disintegration bath containing sodium acetate at pH 4.5. None of the tablets exhibited signs of bloating, cracks, or fissures. Acid uptake values of coated placebos were less than 5.0%.

What is claimed is:

1. A dry, enteric, film-coating composition comprising of an enteric polymer micronized with a detackifier.

2. The dry, enteric, film-coating composition of claim 1, wherein the enteric polymer is an acrylic resin comprising:
a) from 20 to 85 percent by weight of at least one alkyl acrylate or alkyl methacrylate moiety,
b) from 80 to 15 percent by weight of at least one vinyl or vinylidene moiety having a carboxylic group, and
c) from 0 to 30 percent by weight of at least one other vinyl or vinylidene moiety copolymerizable with a) or b).

3. The dry, enteric, film-coating composition of claim 2, wherein said acrylic resin comprises a copolymer of ethyl acrylate and methacrylic acid.

4. The dry, enteric, film-coating composition of claim 1, wherein said enteric polymer is present in an amount of from about 40 to about 70% by weight.

5. The dry, enteric, film-coating composition of claim 1, wherein said enteric polymer is present in an amount of from about 45 to about 65% by weight.

6. The dry, enteric, film-coating composition of claim 1, further comprising a second detackifier which is not a part of the micronized blend of said enteric polymer and said detackifier.

7. The dry, enteric, film-coating composition of claim 1, wherein the total amount of the detackifier and second detackifier is from about 0.1 to about 35% by weight.

8. The dry, enteric, film-coating composition of claim 1, wherein the total amount of the detackifier and second detackifier is from about 15 to about 30% by weight.

9. The dry, enteric, film-coating composition of claim 1, wherein the ratio of the enteric polymer to the detackifier is from about 2:1 to about 99:1.

10. The dry, enteric, film-coating composition of claim 9, wherein the ratio of the enteric polymer to the detackifier is from about 3:1 to about 20:1.

11. The dry, enteric, film-coating composition of claim 1, wherein the detackifier is selected from the group consisting of talc, silicon dioxide, silica gel, fumed silica, glyceryl monostearate, kaolin and mixtures thereof.

12. The dry, enteric, film-coating composition of claim 1, wherein the detackifier comprises talc.

13. The dry, enteric, film-coating composition of claim 1, further comprising a plasticizer.

14. The dry, enteric, film-coating composition of claim 13, wherein said plasticizer is selected from the group consisting of triethylcitrate, triacetin, propylene glycol, glyceryltriacetate, acetyltriethylcitrate, acetyltriethylcitrate, dibutyl sebacate, diethylphthalate, polyethylene glycols, glycerin, dibutylphthalate, castor oil, copolymers of propylene oxide and ethylene oxide and mixtures thereof.

15. The dry, enteric, film-coating composition of claim 13, wherein the plasticizer is present in an amount of from about 5 to about 25% by weight.

16. The dry, enteric, film-coating composition of claim 1, wherein the average particle size of the micronized enteric polymer-detackifier mixture is from about 0.1 to about 50 microns.

17. The dry, enteric, film-coating composition of claim 16, wherein the average particle size of the micronized enteric polymer-detackifier mixture is from about 5 to about 15 microns.

18. The dry, enteric, film-coating composition of claim 1, further comprising one or more of a flow aid, a surfactant, a pigment, an anti-agglomerating agent and a secondary film-former.

19. The dry, enteric, film-coating composition of claim 18, wherein the pigment is selected from the group consisting of FD&C and D&C lakes, titanium dioxide, magnesium carbonate, talc, pyrogenic silica, iron oxides, channel black, riboflavin, carmine 40, curcumin, annatto, insoluble dyes, pearlescent pigments based on mica and/or titanium dioxide and mixtures thereof.

20. The dry, enteric, film-coating composition of claim 18, wherein the flow aid is silica.

21. The dry, enteric, film-coating composition of claim 18, wherein the surfactant is selected from the group consisting of sodium lauryl sulfate, dioctyl sodium sulfosuccinate, polysorbates, or mixtures thereof.

22. The dry, enteric, film-coating composition of claim 18, wherein the anti-agglomerating agent is kaolin.

23. The dry, enteric, film-coating composition of claim 2, wherein the alkyl acrylate is ethyl acrylate and the vinyl or vinylidene moiety having a carboxylic acid group capable of salt formation is methacrylic acid.

24. The dry, enteric, film-coating composition of claim 18, wherein the secondary film former is xanthan gum, sodium alginate, propylene glycol alginate, hydroxypropylmethyl-cellulose (HPMC), hydroxyethyl-cellulose (HEC), sodium carboxymethylcellulose (sodium CMC), polyvinylpyrrolidone (PVP), Konjac flour, carrageenan, other film-forming polymer and mixtures thereof.

25. A film-coating dispersion comprising the enteric, film-coating composition of claim 1 and water.

26. The film-coating dispersion of claim 25, further comprising a plasticizer added separately to the dispersion after the film-coating composition of claim 1 has been added to the water.

27. The film-coating dispersion of claim 26, wherein the plasticizer is selected from the group consisting of triethylcitrate, triacetin, propylene glycol, glyceryltriacetate, acetyltriethylcitrate, acetyltriethylcitrate, dibutyl sebacate, diethylphthalate, polyethylene glycols, glycerin, dibutylphthalate, castor oil, copolymers of propylene oxide and ethylene oxide and mixtures thereof.

28. The film-coating dispersion of claim 25, wherein the concentration of the film-coating composition in water is from about 10 to about 20% (w/w).

29. The film-coating dispersion of claim 25, wherein the concentration of the film-coating composition in water is from about 15 to about 20% (w/w).

30. The film-coating dispersion of claim 26, wherein the concentration of the plasticizer in water is from about 0.5 to about 6 % (w/w).

31. A method of making the dry, enteric, film-coating composition of claim 1, comprising miconizing a blend of an enteric polymer and a detackifier.

32. The method of claim 31, further comprising mixing the micronized blend of said enteric polymer and detackifier with a film coating mixture containing a second detackifier and optionally, one or more of a flow aid, a surfactant, an anti-agglomerating agent, a secondary film-former and a pigment.

33. A film-coated, orally-ingestible substrate, resistant to disintegration in pH 4.5 media, comprising
a) a substrate containing one or more medicaments; and
b) a film-coating comprised of the composition of claim 1.

34. A method of making an aqueous coating dispersion for use in pharmaceuticals, confectionery and food, comprising dispersing the composition of claim 1 in water.

35. The method of claim 34, further comprising the step of adding a plasticizer to the dispersion separately.

36. A method of coating substrates such as orally-ingestible substrates with a film coating comprising, providing an aqueous film coating dispersion of claim 25 and applying an effective amount of said coating dispersion onto said substrates to form a film coating on said substrates, and drying the film coating on said substrates.
